(19) United States
(12) Patent Application Publication
(10) Pub. No.: US 2023/0048615 A1
(43) Pub. Date: Feb. 16, 2023

(54) COMPOSITION CONTAINING FLUORINE OIL

(71) Applicant: DAIKIN INDUSTRIES, LTD., Osaka (JP)

(72) Inventors: Tsubasa NAKAUE, Osaka (JP); Daisuke KARUBE, Osaka (JP); Asako YOSHIYAMA, Osaka (JP)

(73) Assignee: DAIKIN INDUSTRIES, LTD., Osaka (JP)

(21) Appl. No.: 17/975,900

(22) Filed: Oct. 28, 2022

Related U.S. Application Data
(63) Continuation of application No. PCT/JP2021/030945, filed on Aug. 24, 2021.

(30) Foreign Application Priority Data
Aug. 31, 2020 (JP) 2020-146120

(51) Int. Cl. C07C 43/12; C09D 201/04; C11D 7/30; C09D 7/63

(52) U.S. Cl. CPC C07C 43/12; C09D 201/04; C11D 7/30; C09D 7/63

(57) ABSTRACT

The present disclosure provides a composition containing (A) 1,1,1,3,3,3-hexafluoroisopropyl methyl ether (HFE-356mmz) and/or 1,1,2,3,3,3-hexafluoropropyl methyl ether (HFE-356mec), and (B) a fluorine oil.

### TECHNICAL FIELD

The present disclosure relates to a composition containing a fluorine oil.

### BACKGROUND ART

Hydrofluoroethers (HFEs) have attracted attention as alternatives to chlorofluorocarbons (CFCs) and hydrochlorofluorocarbons (HCFCs) because of their low global warming potential (GWP), low ozone depletion potential (ODP), and low toxicity.

PTL 1 discloses a cleaning agent containing an azeotropic mixture-like composition containing 1,1,1,3,3,3-hexafluoroisopropyl methyl ether, which is an HFE, and hexafluoroisopropanol.

PTL 2 discloses that an azeotrope-like composition containing 1,1,1,2,3,3-hexafluoro-3-methoxy-propane, which is another HFE, and 1-bromopropane is usable in cleaning processes as a refrigerant.

### CITATION LIST

### Patent Literature

PTL 1: JP2015-143359A
PTL 2: JP2011-506681A

### SUMMARY

The present disclosure includes the subject matter described in the following.

A composition comprising
(A) 1,1,1,3,3,3-hexafluoroisopropyl methyl ether (HFE-356mmz) and/or 1,1,2,3,3,3-hexafluoropropyl methyl ether (HFE-356mec), and
(B) a fluorine oil.

### ADVANTAGEOUS EFFECTS

The present disclosure provides a novel composition containing HFE-356mmz and/or HFE-356mec and a fluorine oil. The present disclosure also provides a solvent composition or cleaning composition containing HFE-356mmz and/or HFE-356mec for removing a fluorine oil.

### DESCRIPTION OF EMBODIMENTS

The present disclosure includes the following embodiments.

The composition according to the present disclosure contains component (A) and component (B). In the present disclosure, component (A) is 1,1,1,3,3,3-hexafluoroisopropyl methyl ether (HFE-356mmz) and/or 1,1,2,3,3,3-hexafluoropropyl methyl ether (HFE-356mec). In the present disclosure, component (B) is a fluorine oil.

The fluorine oil, which is component (B), is preferably at least one compound selected from the group consisting of perfluoropolyether compounds, compounds containing a perfluoropolyether group and a functional group, and low-molecular polymers of chlorotrifluoroethylene.

Examples of perfluoropolyether compounds include compounds having a constituent unit represented by the following formula (1).

—(OCₙF₂ₙ)— wherein n represents 1,2,3, or 4. Formula (1):

Commercial products of perfluoropolyether compounds include Krytox (registered trademark) GPL oil, 143 oil, vacuum pump oil (trade names, produced by Chemours), Demnum S-20, Demnum S-65, Demnum S-200 (trade names, produced by Daikin Industries, Ltd.), Fomblin M, Fomblin Z, Fomblin Y (trade names, produced by Solvay Specialty Polymers Japan K.K.), and BARRIERTA (trade name, produced by NOK Klüber Co., Ltd.).

Examples of compounds containing a perfluoropolyether group and a functional group include compounds represented by the following formula (2).

(Rf²-PEPE²-Z²)ₓ-A Formula (2):

wherein

PFPE² represents independently in each occurrence a group represented by formula:—(OC₆F₁₂)ₐ—(OC₅F₁₀)_{b}—(OC₄F₆)_{c}—(OC₃F₆)_{d}—(OC₂F₄)ₑ—(OCF₂)_{f}—

(wherein a, b, c, d, e, and f each independently represent an integer of 0 or more and 200 or less, the sum of a, b, c, d, e, and f is at least 1, and the order of the repeating units in parentheses accompanied by a, b, c, d, e, or f is optional in the formula);

Rf² represents independently in each occurrence a C₁₋₁₆ alkyl group optionally substituted with one or more fluorine atoms;

Z² represents a single bond or a divalent organic group;

x represents 1 or 2;

A represents independently in each occurrence —OH, —SH, —NR₂, —COOR, —SO₃H, —PO(OR³)₂, or —SO₂ (OR³); and

R represents a hydrogen atom or a hydrocarbon group.

Examples of compounds containing a perfluoropolyether group and a functional group represented by formula (2) include carboxylic acid compounds containing a perfluoropolyether group (monocarboxylic acid compounds containing a perfluoropolyether group or dicarboxylic acid compounds containing a perfluoropolyether group), ester compounds containing a perfluoropolyether group, silane compounds containing a perfluoropolyether group, hydroxyl compounds containing a perfluoropolyether group, amino compounds containing a perfluoropolyether group, amide compounds containing a perfluoropolyether group, thiol compounds containing a perfluoropolyether group, sulfonic acid compounds containing a perfluoropolyether group, phosphonic acid ester compounds containing a perfluoropolyether group, and sulfonic acid ester compounds containing a perfluoropolyether group. Of these compounds containing a perfluoropolyether group and a functional group, monocarboxylic acid compounds containing a perfluoropolyether group, dicarboxylic acid compounds containing a perfluoropolyether group, or silane compounds containing a perfluoropolyether group are preferable.

Examples of carboxylic acid compounds containing a perfluoropolyether group (monocarboxylic acid compounds containing a perfluoropolyether group or dicarboxylic acid compounds containing a perfluoropolyether group), ester compounds containing a perfluoropolyether group, hydroxyl compounds containing a perfluoropolyether group, or ester compounds containing a perfluoropolyether group include compounds represented by the following formula (3).

X1-O—Rf—Y Formula (3):

wherein —Rf represents a perfluoropolyoxyalkylene chain with a number average molecular weight of 500 to 10,000 containing a unit of (C₂F₄O), (CF₂O), a (C₃F₆O) type (wherein the (C₃F₆O) unit can be a unit represented by formula —(CF₂CF(CF₃)O) or (CF(CF₃)CF₂O)—), (CF₂(CF₂)_{z}CF₂O)— (wherein z represents an integer of 1 or 2), or —CR4R5CF₂CF₂O— (wherein R4 and R5 are identical or different and selected from H, Cl, or, for example, C₁₋₄ perfluoroalkyl) with the unit statistically distributed along the main chain; and —X1 and Y is a terminal group and contains at least one group selected from the group consisting of a carboxyl group, a hydroxy group, an amino group, and an ester group, provided that when one terminal group contains only one of these groups, the other terminal group contains a CF₃ group.

Specific examples of dicarboxylic acid compounds containing a perfluoropolyether group include HOOCCF₂—O—[(CF₂CF₂O)₂₈—(CF₂O)₂₂]—CF₂COOH produced by Daikin Industries, Ltd. Specific examples of monocarboxylic acid compounds containing a perfluoropolyether group include CF₃—O—[(CF₂CF₂O)₂₈—(CF₂O)₂₂]—CF₂COOH produced by Daikin Industries, Ltd. Examples of commercial products of silane compounds containing a perfluoropolyether group include KY-100 series produced by Shin-Etsu Chemical Co., Ltd. (e.g., KY-178, KY-185, KY-195), OPTOOL (registered trademark) DSX, OPTOOL (registered trademark) AES, OPTOOL (registered trademark) UF503, and OPTOOL (registered trademark) UD509 produced by Daikin Industries, Ltd., and Afluid (registered trademark) S550 produced by AGC Inc. Specific examples of silane compounds containing a perfluoropolyether group include OPTOOL (registered trademark) UD500 produced by Daikin Industries, Ltd.

In the present specification, the “low-molecular polymer of chlorotrifluoroethylene” refers to a polymer of chlorotrifluoroethylene with a number average molecular weight of 10,000 or less.

Commercial products of low-molecular polymers of chlorotrifluoroethylene include DAIFLOIL #1, DAIFLOIL #3, DAIFLOIL #10, DAIFLOIL #20, DAIFLOIL #50, and DAIFLOIL #100 (trade names, produced by Daikin Industries, Ltd.).

In the present disclosure, from the standpoint of achieving sufficient lubricity and sufficient coating film strength, component (B) for use is preferably a fluorine oil with a kinematic viscosity at 20° C. of 30 cst or more and 2000 cst or less or a fluorine oil with a kinematic viscosity at 25° C. of 100 cst or more and 2000 cst or less. In the present disclosure, from the standpoint of further increasing lubricity and coating film strength, component (B) for use is more preferably a fluorine oil with a kinematic viscosity at 20° C. of 50 cst or more and 1500 cst or less or a fluorine oil with a kinematic viscosity at 25° C. of 300 cst or more and 1500 cst or less.

In the present disclosure, the fluorine oil, which is component (B), is more preferably at least one compound selected from the group consisting of perfluoropolyether compounds, monocarboxylic acid compounds containing a perfluoropolyether group, dicarboxylic acid compounds containing a perfluoropolyether group, silane compounds containing a perfluoropolyether group, and low-molecular polymers of chlorotrifluoroethylene.

In the present disclosure, preferably, the content of component (A) is 1 mass % or more and 99.99 mass % or less, and the content of component (B) is 0.01 mass % or more and 99 mass % or less based on the total amount of component (A) and component (B). When the content of component (A) and the content of component (B) are within these ranges, component (A) and component (B) in the composition exhibit excellent solubility, resulting in a uniform composition. In the present specification, the total amount of component (A) and component (B) means that the sum of the content of component (A) and the content of component (B) in the composition is 100 mass %.

In the present disclosure, more preferably, the content of component (A) is 1 mass % or more and 99.9 mass % or less, and the content of component (B) is 0.1 mass % or more and 99 mass % or less, based on the total amount of component (A) and component (B).

In the present disclosure, still more preferably, the content of component (A) is 20 mass % or more and 99.5 mass % or less, and the content of component (B) is 0.5 mass % or more and 80 mass % or less, based on the total amount of component (A) and component (B).

In the present disclosure, particularly preferably, the content of component (A) is 50 mass % or more and 99 mass % or less, and the content of component (B) is 1 mass % or more and 50 mass % or less, based on the total amount of component (A) and component (B).

As described in Comparative Example 1 later, from the standpoint of dissolving HFE-356mmz in a fluorine oil, the composition of the present disclosure preferably contains no HFIP.

In the present disclosure, the total amount of component (A) and component (B) in the total amount of the composition taken as 100 mass % is preferably more than 50 mass %, more preferably 80 mass % or more, still more preferably 90 mass % or more, yet more preferably 99 mass % or more, and particularly preferably 99.5 mass % or more.

It is the most preferable that the composition of the present disclosure consists of component (A) and component (B) (with the proviso that, unavoidable impurities other than component (A) and component (B) are allowed to be present).

It has been unknown that HFE-356mmz and HFE-356mec each exhibit excellent miscibility with a fluorine oil. However, the inventors of the present disclosure, as a result of trial and error of numerous combinations of HFE and oil, surprisingly found that a composition of HFE-356mmz or HFE-356mec with a fluorine oil does not become turbid even when the content of the fluorine oil is set to such a high concentration as described above and becomes transparent due to the high miscibility of HFE-356mmz or HFE-356mec with the fluorine oil.

In the present specification, “transparent” means that the composition is uniform and not turbid.

The composition of the present disclosure can be suitably used as a coating agent because of the excellent miscibility of HFE-356mmz or HFE-356mec with a fluorine oil and the moderately high boiling point of these mixtures.

To achieve the excellent miscibility, the composition of the present disclosure has a boiling point of preferably 40° C. or more and 100° C. or less, and more preferably 50° C. or more and 90° C. or less. When the composition of the present disclosure having a boiling point within these temperature ranges is used in a coating agent, the composition will exhibit excellent solubility with limited evaporation.

In the present specification, the term “coating agent” refers to a mixture that exists in a liquid form at room temperature and that is applied to an article to form a coating film mainly for the purpose of, for example, protecting or imparting luster to the surface of the article.

Another aspect of the present disclosure is a coating method including applying the composition of the present disclosure.

Another aspect of the present disclosure is a solvent composition or cleaning composition for removing a fluorine oil, containing HFE-356mmz and/or HFE-356mec.

The fluorine oil to be removed is preferably at least one compound selected from the group consisting of perfluoropolyether compounds, compounds containing a perfluoropolyether group and a functional group, and low-molecular polymers of chlorotrifluoroethylene. Of these, perfluoropolyether compounds are particularly preferable. The perfluoropolyether compounds, the compounds containing a perfluoropolyether group and a functional group, and the low-molecular polymers of chlorotrifluoroethylene are as described above.

### Optional Additives

The composition of the present disclosure may contain, in addition to HFE-356mmz or HFE-356mec, at least one member selected from the group consisting of additional solvents other than HFE-356mmz or HFE-356mec, antioxidants, stabilizers, preservatives, and surfactants.

Additional solvents for use can be selected from a wide range of solvents such as alkane compounds, alkene compounds, chloroalkane compounds, chloroalkene compounds, hydrofluorcalkane compounds, fluoroalkene compounds, chlorofluoroalkene compounds, and alcohol compounds, according to the properties to be added. However, hexafluoroisopropanol (HFIP) is not preferable as such an additional solvent in the present disclosure. For example, for the purpose of dissolving mineral oil, 1,2-dichloroethylene (chloroalkene compound) or 1,2-dichloro-3,3,3-trifluoropropene (chlorofluoroalkene compound) can be added. Isopropyl alcohol (IPA), 1-butanol, or 2,2,3,3,4,4,5,5,6,6,7,7-dodecafluoro-1-heptanol (alcohol compounds), for example, can also be added. In the present disclosure, additional solvents are preferably an alcohol compound. The alcohol compound is preferably at least one member selected from the group consisting of isopropyl alcohol (IPA), 1-butanol, and 2,2,3,3,4,4,5,5,6,6,7,7-dodecafluoro-1-heptanol, and more preferably isopropyl alcohol (IPA). If an additional solvent is added, the composition of the present disclosure contains such an additional solvent in an amount of preferably 0.1 mass % or more and less than 50 mass %, more preferably 0.5 mass % or more and 20 mass % or less, and still more preferably 1 mass % or more and 10 mass % or less, based on 100 mass % of the composition.

The stabilizers become functional as an acid acceptor or an antioxidant by exerting a stabilization effect. Major stabilization effects include the effect of preventing the decomposition of HFE-356mmz or HFE-356mec by capturing radicals generated in a system, and the acid-accepting effect of preventing further decomposition of HFE-356mmz or HFE-356mec caused by an acid by capturing the acid generated in a system. Such stabilizers for use are selected from a wide range of known stabilizers. In particular, from the standpoint of effectively reducing metal corrosion by the composition, the stabilizer for use is preferably at least one stabilizer selected from the group consisting of unsaturated alcohol-based stabilizers, nitro-based stabilizers, amine-based stabilizers, phenol-based stabilizers, and epoxy-based stabilizers.

The unsaturated alcohol-based stabilizer for use can be selected from a wide range of known such stabilizers. For example, the unsaturated alcohol-based stabilizer for use is at least one member selected from the group consisting of 3-buten-2-ol, 2-buten-1-ol, 4-propen-1-ol, 1-propen-3-ol, 2-methyl-3-buten-2-ol, 3-methyl-3-buten-2-ol, 3-methyl-2-buten-1-ol, 2-hexen-1-ol, 2,4-hexadien-1-ol, and oleyl alcohol.

The nitro-based stabilizer for use can be selected from a wide range of known such stabilizers. Examples of aliphatic nitro compounds include nitromethane, nitroethane, 1-nitropropane, and 2-nitropropane. For example, the aromatic nitro compound for use is at least one member selected from the group consisting of nitrobenzene, o-, m-, or p-dinitrobenzene, o-, m-, or p-nitrotoluene, dimethyl nitrobenzene, m-nitroacetophenone, o-, m-, or p-nitrophenol, o-nitroanisole, m-nitroanisole, and p-nitroanisole.

The amine-based stabilizer for use can be selected from a wide range of known such stabilizers. For example, the amine-based stabilizer for use is at least one member selected from the group consisting of pentylamine, hexylamine, diisopropylamine, diisobutylamine, di-n-propylamine, diallylamine, triethylamine, N-methylaniline, pyridine, morpholine, N-methylmorpholine, triallylamine, allylamine, α-methylbenzylamine, dimethylamine, trimethylamine, ethylamine, diethylamine, triethylamine, propylamine, isopropylamine, dipropylamine, tripropylamine, butylamine, isobutylamine, dibutylamine, tributylamine, dibenzylamine, tribenzylamine, 2-ethylhexylamine, aniline, N,N-dimethylaniline, N,N-diethylaniline, ethylenediamine, propylenediamine, diethylenetriamine, tetraethylenepentamine, benzylamine, dibenzylamine, diphenylamine, and diethylhydroxylamine.

The phenol-based stabilizer for use can be selected from a wide range of known such stabilizers. For example, the phenol-based stabilizer for use is at least one member selected from the group consisting of 2,6-ditertiarybutyl-4-methylphenol, 3-cresol, phenol, 1,2-benzenediol, 2-isopropyl-5-methylphenol, and 2-methoxyphenol.

The epoxy-based stabilizer for use can be selected from a wide range of known such stabilizers. For example, the epoxy-based stabilizer for use is at least one member selected from the group consisting of butylene oxide, 1,2-propylene oxide, 1,2-butylene oxide, butyl glycidyl ether, diethylene glycol diglycidyl ether, and 1,2-epoxy-3-phenoxy propane.

From the standpoint of more effectively preventing the decomposition of HFE-356mmz or HFE-356mec caused by various factors by using a combination of stabilizers having different stabilization effects, the stabilizer is preferably composed of the above-described epoxy-based stabilizer and at least one member selected from the group consisting of the unsaturated alcohol-based stabilizers, nitro-based stabilizers, and phenol-based stabilizers.

From the standpoint of more effectively limiting acid liberation from HFE-356mmz or HFE-356mec and reducing metal corrosion caused by the composition in a liquid form, the content of the stabilizer in the composition taken as 100 mass % is preferably 0.0001 mass % or more, and more preferably 0.01 mass % or more. However, in consideration of avoiding unfavorable changes in physical properties of the composition in a liquid form due to an excessively added stabilizer, the content of the stabilizer in the composition taken as 100 mass % is preferably 10 mass % or less, and more preferably 5 mass % or less.

Another aspect of the present disclosure is a cleaning method including removing a fluorine oil by using a cleaning agent containing HFE-356mmz and/or HFE-356mec.

The fluorine oil to be removed is preferably at least one compound selected from the group consisting of perfluoropolyether compounds, compounds containing a perfluoropolyether group and a functional group, and low-molecular polymers of chlorotrifluoroethylene. Of these, perfluoropolyether compounds are particularly preferable. The perfluoropolyether compounds, the compounds containing a perfluoropolyether group and a functional group, and the low-molecular polymers of chlorotrifluoroethylene are as described above.

Another aspect of the present disclosure is a dissolution method including dissolving a fluorine oil by using a solvent containing HFE-356mmz and/or HFE-356mec.

The fluorine oil to be dissolved is preferably at least one compound selected from the group consisting of perfluoropolyether compounds, compounds containing a perfluoropolyether group and a functional group, and low-molecular polymers of chlorotrifluoroethylene. Of these, perfluoropolyether compounds are particularly preferable. The perfluoropolyether compounds, the compounds containing a perfluoropolyether group and a functional group, and the low-molecular polymers of chlorotrifluoroethylene are as described above.

A composition containing HFE-356mmz and/or HFE-356mec (100 parts by mass) mixed with preferably 20 parts by mass or less of a fluorine oil is usable as a solvent or cleaning agent. Additionally, for example, after the composition is used as a solvent or cleaning agent, the composition containing HFE-356mmz and/or HFE-356mec that remains mixed with a fluorine oil can be used as a solvent or cleaning agent again.

### EXAMPLES

Embodiments of the present disclosure are described in more detail with reference to the following Examples. However, the present disclosure is not limited to these Examples.

The following fluorine oils were used in the Examples and Comparative Examples.
Fluorine Oil (A1): Demnum 5-20, produced by Daikin Industries, Ltd., a perfluoropolyether compound (kinematic viscosity at 20° C.=53 cst)
Fluorine Oil (A2): Demnum S-65, produced by Daikin Industries, Ltd., a perfluoropolyether compound (kinematic viscosity at 20° C.=150 cst)
Fluorine Oil (A3): Demnum S-200, produced by Daikin Industries, Ltd., a perfluoropolyether compound (kinematic viscosity at 20° C.=500 cst)
Fluorine Oil (A4): BARRIERTA J100, produced by NOK Klüber Co., Ltd, a perfluoropolyether compound (kinematic viscosity at 20° C.=280 cst)
Fluorine Oil (A5): Fomblin M30, produced by Solvay Specialty Polymers Japan K.K., a perfluoropolyether compound (kinematic viscosity at 20° C.=280 cst)
Fluorine Oil (A6): Fomblin Y140/13, produced by Solvay Specialty Polymers Japan K.K., a perfluoropolyether compound (kinematic viscosity at 20° C.=1400 cst)
Fluorine Oil (A7): OPTOOL UD500, produced by Daikin Industries, Ltd., a silane compound containing a perfluoropolyether group
Fluorine Oil (A8): DAIFLOIL #20, produced by Daikin Industries, Ltd., a low-molecular polymer of chlorotrifluoroethylene (kinematic viscosity at 25° C.=350 to 1500 cst)
Fluorine Oil (A9): HOOCCF₂—O—[(CF₂CF₂O)₂₈—(CF₂O)₂₂]—CF₂COOH, produced by Daikin Industries, Ltd., a dicarboxylic acid compound containing a perfluoropolyether group
Fluorine Oil (A10): CF₃—O—[(CF₂CF₂O)₂₈—(CF₂O)₂₂]—CF₂COOH, produced by Daikin Industries, Ltd., a monocarboxylic acid compound containing a perfluoropolyether group

### Miscibility Test

### Example 1

1,1,1,3,3,3-Hexafluoroisopropyl methyl ether (HFE-356mmz) and fluorine oil (A1) were placed in a glass test tube to prepare a composition. Whether HFE-356mmz and fluorine oil (A1) were dissolved into each other at 25° C. was observed. The content of each component in the composition was HFE-356mmz: 50 mass %, fluorine oil (A1): 50 mass %. The glass test tube was observed, and the composition of HFE-356mmz and fluorine oil (A1) was found to be a single layer and stay in a transparent liquid form. Thus, fluorine oil (A1) was confirmed to have been completely dissolved in HFE-356mmz.

### Example 2

A composition was prepared under the same conditions as in Example 1, except that the content of HFE-356mmz was 20 mass %, and the content of fluorine oil (A1) was 80 mass %. Whether HFE-356mmz and fluorine oil (A1) were dissolved into each other at 25° C. was observed. The glass test tube was observed, and the composition of HFE-356mmz and fluorine oil (A1) was found to be a single layer and stay in a transparent liquid form. Thus, fluorine oil (A1) was confirmed to have been completely dissolved in HFE-356mmz.

### Example 3

A composition was prepared under the same conditions as in Example 1, except that the content of HFE-356mmz was 80 mass %, and the content of fluorine oil (A1) was 20 mass %. Whether HFE-356mmz and fluorine oil (A1) were dissolved into each other at 25° C. was observed. The glass test tube was observed, and the composition of HFE-356mmz and fluorine oil (A1) was found to be a single layer and stay in a transparent liquid form. Thus, fluorine oil (A1) was confirmed to have been completely dissolved in HFE-356mmz.

### Example 4

A composition was prepared under the same conditions as in Example 1, except that fluorine oil (A2) was used instead of fluorine oil (A1). Whether HFE-356mmz and fluorine oil (A2) were dissolved into each other at 25° C. was observed. The glass test tube was observed, and the composition of HFE-356mmz and fluorine oil (A2) was found to be a single layer and stay in a transparent liquid form. Thus, fluorine oil (A2) was confirmed to have been completely dissolved in HFE-356mmz.

### Example 5

A composition was prepared under the same conditions as in Example 1, except that fluorine oil (A3) was used instead of fluorine oil (A1). Whether HFE-356mmz and fluorine oil (A3) were dissolved into each other at 25° C. was observed. The glass test tube was observed, and the composition of HFE-356mmz and fluorine oil (A3) was found to be a single layer and stay in a transparent liquid form. Thus, fluorine oil (A3) was confirmed to have been completely dissolved in HFE-356mmz.

### Example 6

A composition was prepared under the same conditions as in Example 1, except that fluorine oil (A4) was used instead of fluorine oil (A1). Whether HFE-356mmz and fluorine oil (A4) were dissolved into each other at 25° C. was observed. The glass test tube was observed, and the composition of HFE-356mmz and fluorine oil (A4) was found to be a single layer and stay in a transparent liquid form. Thus, fluorine oil (A4) was confirmed to have been completely dissolved in HFE-356mmz.

### Example 7

A composition was prepared under the same conditions as in Example 1, except that fluorine oil (A5) was used instead of fluorine oil (A1). Whether HFE-356mmz and fluorine oil (A5) were dissolved into each other at 25° C. was observed. The glass test tube was observed, and the composition of HFE-356mmz and fluorine oil (A5) was found to be a single layer and stay in a transparent liquid form. Thus, fluorine oil (A5) was confirmed to have been completely dissolved in HFE-356mmz.

### Example 8

A composition was prepared under the same conditions as in Example 1, except that fluorine oil (A6) was used instead of fluorine oil (A1). Whether HFE-356mmz and fluorine oil (A6) were dissolved into each other at 25° C. was observed. The glass test tube was observed, and the composition of HFE-356mmz and fluorine oil (A6) was found to be a single layer and stay in a transparent liquid form. Thus, fluorine oil (A6) was confirmed to have been completely dissolved in HFE-356mmz.

### Example 9

A composition was prepared under the same conditions as in Example 1, except that fluorine oil (A7) was used instead of fluorine oil (A1). Whether HFE-356mmz and fluorine oil (A7) were dissolved into each other at 25° C. was observed. The glass test tube was observed, and the composition of HFE-356mmz and fluorine oil (A7) was found to be a single layer and stay in a transparent liquid form. Thus, fluorine oil (A7) was confirmed to have been completely dissolved in HFE-356mmz.

### Example 10

A composition was prepared under the same conditions as in Example 1, except that fluorine oil (A8) was used instead of fluorine oil (A1). Whether HFE-356mmz and fluorine oil (A8) were dissolved into each other at 25° C. was observed. The glass test tube was observed, and the composition of HFE-356mmz and fluorine oil (A8) was found to be a single layer and stay in a transparent liquid form. Thus, fluorine oil (A8) was confirmed to have been completely dissolved in HFE-356mmz.

### Example 11

A composition was prepared under the same conditions as in Example 1, except that fluorine oil (A9) was used instead of fluorine oil (A1). Whether HFE-356mmz and fluorine oil (A9) were dissolved into each other at 25° C. was observed. The glass test tube was observed, and the composition of HFE-356mmz and fluorine oil (A9) was found to be a single layer and stay in a transparent liquid form. Thus, fluorine oil (A9) was confirmed to have been completely dissolved in HFE-356mmz.

### Example 12

A composition was prepared under the same conditions as in Example 1, except that fluorine oil (A10) was used instead of fluorine oil (A1). Whether HFE-356mmz and fluorine oil (A10) were dissolved into each other at 25° C. was observed. The glass test tube was observed, and the composition of HFE-356mmz and fluorine oil (A10) was found to be a single layer and stay in a transparent liquid form. Thus, fluorine oil (A10) was confirmed to have been completely dissolved in HFE-356mmz.

### Example 13

1,1,2,3,3,3-Hexafluoropropyl methyl ether (HFE-356mec) and fluorine oil (A1) were placed in a glass test tube to prepare a composition. Whether HFE-356mec and fluorine oil were dissolved into each other at 25° C. was observed. The content of each component in the composition was HFE-356mec: 50 mass %, fluorine oil (A1): 50 mass %. The glass test tube was observed, and the composition of HFE-356mec and fluorine oil (A1) was found to be a single layer and stay in a transparent liquid form. Thus, fluorine oil (A1) was confirmed to have been completely dissolved in HFE-356mec.

### Example 14

A composition was prepared under the same conditions as in Example 13, except that fluorine oil (A2) was used instead of fluorine oil (A1). Whether HFE-356mec and fluorine oil (A2) were dissolved into each other at 25° C. was observed. The glass test tube was observed, and the composition of HFE-356mec and fluorine oil (A2) was found to be a single layer and stay in a transparent liquid form. Thus, fluorine oil (A2) was confirmed to have been completely dissolved in HFE-356mec.

### Example 15

A composition was prepared under the same conditions as in Example 13, except that fluorine oil (A4) was used instead of fluorine oil (A1). Whether HFE-356mec and fluorine oil (A4) were dissolved into each other at 25° C. was observed. The glass test tube was observed, and the composition of HFE-356mec and fluorine oil (A4) was found to be a single layer and stay in a transparent liquid form. Thus, fluorine oil (A4) was confirmed to have been completely dissolved in HFE-356mec.

### Example 16

A composition was prepared under the same conditions as in Example 13, except that fluorine oil (A5) was used instead of fluorine oil (A1). Whether HFE-356mec and fluorine oil (A5) were dissolved into each other at 25° C. was observed. The glass test tube was observed, and the composition of HFE-356mec and fluorine oil (A5) was found to be a single layer and stay in a transparent liquid form. Thus, fluorine oil (A5) was confirmed to have been completely dissolved in HFE-356mec.

### Example 17

HFE-356mec and fluorine oil (A6) were placed in a glass test tube to prepare a composition. Whether HFE-356mec and the fluorine oil were dissolved into each other at 25° C. was observed. The content of each component in the composition was HFE-356mec: 0.1 mass %, fluorine oil (A6): 99.9 mass %. The glass test tube was observed, and the composition of HFE-356mec and fluorine oil (A6) was found to be a single layer and stay in a transparent liquid form. Thus, fluorine oil (A6) was confirmed to have been completely dissolved in HFE-356mec.

### Example 18

A composition was prepared under the same conditions as in Example 13, except that fluorine oil (A7) was used instead of fluorine oil (A1). Whether HFE-356mec and fluorine oil (A7) were dissolved into each other at 25° C. was observed. The glass test tube was observed, and the composition of HFE-356mec and fluorine oil (A7) was found to be a single layer and stay in a transparent liquid form. Thus, fluorine oil (A7) was confirmed to have been completely dissolved in HFE-356mec.

### Example 19

A composition was prepared under the same conditions as in Example 13, except that fluorine oil (A8) was used instead of fluorine oil (A1). Whether HFE-356mec and fluorine oil (A8) were dissolved into each other at 25° C. was observed. The glass test tube was observed, and the composition of HFE-356mec and fluorine oil (A8) was found to be a single layer and stay in a transparent liquid form. Thus, fluorine oil (A8) was confirmed to have been completely dissolved in HFE-356mec.

### Example 20

A composition was prepared under the same conditions as in Example 13, except that fluorine oil (A9) was used instead of fluorine oil (A1). Whether HFE-356mec and fluorine oil (A9) were dissolved into each other at 25° C. was observed. The glass test tube was observed, and the composition of HFE-356mec and fluorine oil (A9) was found to be a single layer and stay in a transparent liquid form. Thus, fluorine oil (A9) was confirmed to have been completely dissolved in HFE-356mec.

### Example 21

A composition was prepared under the same conditions as in Example 13, except that fluorine oil (A10) was used instead of fluorine oil (A1). Whether HFE-356mec and fluorine oil (A10) were dissolved into each other at 25° C. was observed. The glass test tube was observed, and the composition of HFE-356mec and fluorine oil (A10) was found to be a single layer and stay in a transparent liquid form. Thus, fluorine oil (A10) was confirmed to have been completely dissolved in HFE-356mec.

### Example 22

HFE-356mec and isopropyl alcohol (IPA) were placed in a glass test tube to prepare a mixture. The content of each component in the mixture was HFE-356mec: 92 mass %, IPA: 8 mass %. Additionally, fluorine oil (A1) was added to the glass test tube such that the compositional percentages of the mixture and fluorine oil (A1) were respectively 95 mass % and 5 mass %. Whether the mixture and fluorine oil (A1) were dissolved into each other at 25° C. was observed. The glass test tube was observed, and the composition of the mixture and fluorine oil (A1) was found to be a single layer and stay in a transparent liquid form. Thus, fluorine oil (A1) was confirmed to have been completely dissolved in the mixture.

### Comparative Example 1

HFE-356mmz and hexafluoroisopropanol (HFIP) were placed in a glass test tube to prepare a mixture. The content of each component in the mixture was HFE-356mmz: 50 mass %, HFIP: 50 mass %. Additionally, fluorine oil (A3) was added to the glass test tube such that the compositional percentages of the mixture and fluorine oil (A3) were respectively 50 mass % and 50 mass %. Whether the mixture and fluorine oil (A3) were dissolved into each other at 25° C. was observed. The glass test tube was observed, and the mixture and fluorine oil (A3) were confirmed to have not been dissolved into each other, and to have separated into two layers.

### Comparative Example 2

HFE-356mmz and HFIP were placed in a glass test tube to prepare a mixture. The content of each component in the mixture was HFE-356mmz: 90 mass %, HFIP: 10 mass %. Additionally, fluorine oil (A3) was added to the glass test tube such that the compositional percentages of the mixture and fluorine oil (A3) were respectively 50 mass % and 50 mass %. Whether the mixture and fluorine oil (A3) were dissolved into each other at 25° C. was observed. The glass test tube was observed, and the mixture and fluorine oil (A3) were confirmed to have not been dissolved into each other, and to have separated into two layers.

The results of Comparative Example 1 and Comparative Example 2 indicate that the mixture of HFE-356mmz and HFIP is not miscible with fluorine oil (A3).

### Comparative Example 3

In this Comparative Example, bis(2,2,2-trifluoroethyl) ether (HFE-356mff2, CF₃CH₂OCH₂CF₃) was used as an HFE compound. HFE-356mff2 is an HFE compound composed of four carbon atoms, six fluorine atoms, four hydrogen atoms, and one oxygen atom, in the same manner as HFE-356mmz and HFE-356mec in the present disclosure.

Bis(2,2,2-trifluoroethyl) ether (HFE-356mff2, CF₃CH₂OCH₂CF₃) and fluorine oil (A2) were placed in a glass test tube to prepare a composition. Whether HFE-356mff2 and fluorine oil (A2) were dissolved into each other at 25° C. was observed. The content of each component in the composition was HFE-356mff2: 50 mass %, fluorine oil (A2): 50 mass %. The glass test tube was observed, and HFE-356mff2 and fluorine oil (A2) were confirmed to have not been dissolved into each other, and to have separated into two layers.

The results of Comparative Example 3 indicate that HFE-356mff2 is not miscible with fluorine oil (A2). It was also confirmed that even HFE compounds composed of the same constituent elements with the same number of those elements, as with HFE-356mmz, HFE-356mec, and HFE-356mff2 (i.e., structural isomers), differ as to whether these compounds can dissolve in fluorine oil or not.

### Comparative Example 4

In this Comparative Example, 1,1,2,2-tetrafluoro-1-(2,2,2-trifluoroethoxy) ethane (HFE-347pcf, CF₂HCF₂OCH₂CF₃) was used as an HFE compound.

HFE-347pcf and fluorine oil (A2) were placed in a glass test tube to prepare a composition. Whether HFE-347pcf and fluorine oil (A2) were dissolved into each other at 25° C. was observed. The content of each component in the composition was HFE-347pcf: 50 mass %, fluorine oil (A2): 50 mass %. The glass test tube was observed, and HFE-347pcf and fluorine oil (A2) were confirmed to have not been dissolved into each other, and to have separated into two layers.

### Coatability Test

### Example 23

HFE-356mmz and fluorine oil (A2) (lubricant) were mixed in a glass test tube to prepare a lubricant solution. The content of each component in the lubricant solution was HFE-356mmz: 97 mass %, fluorine oil (A2): 3 mass %. Subsequently, the lubricant solution was applied to the surface of an aluminum substrate to give an average thickness of 0.5 mm, and dried in air at 24 to 27° C., thereby forming a lubricant coating film on the surface of the aluminum substrate. The lubricant coating film was visually observed and confirmed to be a uniform coating film.

### Stability Test

### Example 24

HFE-356mmz and fluorine oil (A2) were mixed in a glass test tube to prepare a solution. The content of each component in the solution was HFE-356mmz: 50 mass %, fluorine oil (A2): 50 mass %. After the obtained solution was allowed to stand at 25° C. for 30 days, the state of the solution was visually observed. The solution was confirmed to have undergone no changes in color, precipitation formation, etc.

### Cleaning Test

### Example 25

A glass testpiece (50 mm×5 mm×2 mm) was immersed in fluorine oil (A2) for 30 seconds. The glass testpiece was then immersed in 100 ml of HFE-356mmz for 1 minute at 25° C. and pulled out, followed by drying in air at room temperature for 5 minutes. As a result, fluorine oil (A2) remaining on the glass testpiece was confirmed to have been removed.

### Coatability Test

### Example 26

HFE-356mec and fluorine oil (A2) (lubricant) were mixed in a glass test tube to prepare a lubricant solution. The content of each component in the lubricant solution was HFE-356mec: 97 mass %, fluorine oil (A2): 3 mass %. Subsequently, the lubricant solution was applied to the surface of an aluminum substrate to give an average thickness of 0.5 mm and dried in air at 24 to 27° C., thereby forming a lubricant coating film on the surface of the aluminum substrate. The lubricant coating film was visually observed and confirmed to be a uniform coating film.

### Stability Test

### Example 27

HFE-356mec and fluorine oil (A2) were mixed in a glass test tube to prepare a solution. The content of each component in the solution was HFE-356mmz: 50 mass %, fluorine oil (A2): 50 mass %. After the obtained solution was allowed to stand at 25° C. for 30 days, the state of the solution was visually observed. The solution was confirmed to have undergone no changes in color, precipitation formation, etc.

### Cleaning Test

### Example 28

A glass testpiece (50 mm×5 mm×2 mm) was immersed in fluorine oil (A2) for 30 seconds. The glass testpiece was then immersed in 100 ml of HFE-356mec for 1 minute at 25° C. and pulled out, followed by drying in air at room temperature for 5 minutes. As a result, fluorine oil (A2) remaining on the glass testpiece was confirmed to have been removed.

The present disclosure provides the invention according to the following embodiments.

Item 1. A composition comprising
(A) 1,1,1,3,3,3-hexafluoroisopropyl methyl ether (HFE-356mmz) and/or 1,1,2,3,3,3-hexafluoropropyl methyl ether (HFE-356mec), and
(B) a fluorine oil.
   Item 2. The composition according to Item 1, wherein the fluorine oil is at least one compound selected from the group consisting of perfluoropolyether compounds, compounds containing a perfluoropolyether group and a functional group, and low-molecular polymers of chlorotrifluoroethylene.
   Item 3. The composition according to Item 1 or 2, wherein the content of component (A) is 1 mass % or more and 99.99 mass % or less, and the content of component (B) is 0.01 mass % or more and 99 mass % or less, based on the total amount of component (A) and component (B).
   Item 4. The composition according to any one of Items 1 to 3, for use in a coating agent.
   Item 5. A solvent composition or cleaning composition for removing a fluorine oil, the composition comprising 1,1,1,3,3,3-hexafluoroisopropyl methyl ether (HFE-356mmz) and/or 1,1,2,3,3,3-hexafluoropropyl methyl ether (HFE-356mec).
   Item 6. A cleaning method comprising removing a fluorine oil by using a cleaning agent containing 1,1,1,3,3,3-hexafluoroisopropyl methyl ether (HFE-356mmz) and/or 1,1,2,3,3,3-hexafluoropropyl methyl ether (HFE-356mec).
   Item 7. A dissolution method comprising dissolving a fluorine oil by using a solvent containing 1,1,1,3,3,3-hexafluoroisopropyl methyl ether (HFE-356mmz) and/or 1,1,2,3,3,3-hexafluoropropyl methyl ether (HFE-356mec).
   Item 8. A coating method comprising applying the composition of any one of Items 1 to 3.

1-8. (canceled)

9. A composition comprising
(A) 1,1,1,3,3,3-hexafluoroisopropyl methyl ether (HFE-356mmz) and/or 1,1,2,3,3,3-hexafluoropropyl methyl ether (HFE-356mec), and
(B) a fluorine oil.

10. The composition according to claim 9, wherein the fluorine oil is at least one compound selected from the group consisting of perfluoropolyether compounds, compounds containing a perfluoropolyether group and a functional group, and low-molecular polymers of chlorotrifluoroethylene.

11. The composition according to claim 9, wherein the content of component (A) is 1 mass % or more and 99.99 mass % or less, and the content of component (B) is 0.01 mass % or more and 99 mass % or less, based on the total amount of component (A) and component (B).

12. The composition according to claim 10, wherein the content of component (A) is 1 mass % or more and 99.99 mass % or less, and the content of component (B) is 0.01 mass % or more and 99 mass % or less, based on the total amount of component (A) and component (B).

13. The composition according to claim 9, for use in a coating agent.

14. The composition according to claim 10, for use in a coating agent.

15. The composition according to claim 11, for use in a coating agent.

16. The composition according to claim 12, for use in a coating agent.

17. A solvent composition or cleaning composition for removing a fluorine oil, the composition comprising 1,1,1,3,3,3-hexafluoroisopropyl methyl ether (HFE-356mmz) and/or 1,1,2,3,3,3-hexafluoropropyl methyl ether (HFE-356mec).

18. A cleaning method comprising removing a fluorine oil by using a cleaning agent containing 1,1,1,3,3,3-hexafluoroisopropyl methyl ether (HFE-356mmz) and/or 1,1,2,3,3,3-hexafluoropropyl methyl ether (HFE-356mec).

19. A dissolution method comprising dissolving a fluorine oil by using a solvent containing 1,1,1,3,3,3-hexafluoroisopropyl methyl ether (HFE-356mmz) and/or 1,1,2,3,3,3-hexafluoropropyl methyl ether (HFE-356mec).

20. A coating method comprising applying the composition of claim 9.

21. A coating method comprising applying the composition of claim 10.

22. A coating method comprising applying the composition of claim 11.

23. A coating method comprising applying the composition of claim 12.
